(19) United States
(12) Patent Application Publication
(10) Pub. No.: US 2018/0104264 A1
(43) Pub. Date: Apr. 19, 2018

(54) Method for Preparing Lyophilized Organic Solvent-Free Cyclophosphamide

(71) Applicant: KOREA UNITED PHARM. INC., Sejong-si (KR)

(72) Inventors: Won Ho Kang, Seoul (KR); Won Tae Jung, Seoul (KR); Young Ho Lee, Sejong-si (KR); Jung Hoon Kang, Gyeonggi-do (KR); Byuck Ho Kim, Chungcheongnam-do (KR)

(21) Appl. No.: 15/385,033

(22) Filed: Dec. 20, 2016

(30) Foreign Application Priority Data
Oct. 14, 2016 (KR) 10-2016-0133819

(51) Int. Cl. A61K 31/675; A61K 47/02; A61K 47/26; A61K 9/19; A61K 9/00; A61K 9/08

(52) U.S. Cl. CPC A61K 31/675; A61K 47/02; A61K 9/08; A61K 9/19; A61K 9/0019; A61K 47/26

(57) ABSTRACT

The present invention relates to a method for preparing a lyophilized composition with improved stability and solubility, which comprises dissolving cyclophosphamide, sodium chloride, and D-mannitol in water as a solvent in a reaction container at 40° C. to 70° C., and a lyophilized cyclophosphamide composition for injection prepared according to the method.

### CROSS-REFERENCE TO RELATED APPLICATIONS

This application claims priority to Korean Patent Application No. 10-2016-0133819, filed Nov. 12, 2015, the entire contents of which are incorporated herein by reference.

### TECHNICAL FIELD OF THE INVENTION

The present invention relates to a method for preparing a lyophilized composition with improved stability and solubility, which comprises dissolving cyclophosphamide, sodium chloride, and D-mannitol in water as a solvent in a reaction container at 40° C. to 70° C., and a lyophilized cyclophosphamide composition for injection prepared according to the method.

### BACKGROUND OF THE INVENTION

Cyclophosphamide is a synthetic anticancer drug which is chemically belongs to nitrogen mustard, and represented by the formula below:

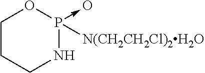

As an example of cyclic phosphoric acid ester amides disclosed in U.S. Pat. No. 3,018,302, granted on Jan. 23, 1962, the chemical name of cyclophosphamide is 2-[bis(2-chloroethyl)amino]tetrahydro-2H-1,3,2-oxazaphosphorin-2-oxide, existing in the form of a monohydrate or an anhydrous form. Although cyclophosphamide is usually used in the form of a monohydrate, it may lose water molecules and become anhydrous at a dry condition having relative humidity below 20 or during a preparation process.

Cyclophosphamide was initially distributed in the form of a monohydrate, and was also provided in a parenteral dose of a pre-mixture consisting of a dry powdered mixture having drugs and sodium chloride sterilized and packed. In addition, cyclophosphamide could be administered not only parenterally but also orally by dissolving the pre-mixture in water prior to administration. However, it was necessary to immediately administer the same since a shelf life of an aqueous solution thereof was only a few hours upon preparation. Meanwhile, a shape of the pre-mixture in the dry powdered state could become hyalinized or sticky during a preparation process and/or a storage period. Additionally, it sometimes had poor properties such as a decrease in the solubility and effect thereof. Such deterioration occurred when a storage period became long or a storage temperature exceeded the recommended upper limit.

Recently, cyclophosphamide has been distributed in the form of a lyophilizate. Although lyophilization had been used for injections of an insoluble drug to an aqueous solution, it was not applied to cyclophosphamide until around 1982. Thereafter, a lyophilized composition comprising cyclophosphamide as an active ingredient and a preparation method thereof were investigated using various additives, but most methods were carried out by using an organic solvent, such as butanol, etc. When even a trace amount of this organic solvent remains in the body, it may cause irritation of the body or exhibit harmful side effects. Therefore, there is an inconvenience in that this organic solvent should remain below a permitted residual standard.

Accordingly, the present inventors have mainly devoted their attention toward discovering a method for preparing a lyophilized cyclophosphamide composition without using an organic solvent. As a result, the present inventors have confirmed the preparation of a lyophilized composition with improved stability and solubility by adjusting a temperature for dissolving cyclophosphamide, an active ingredient, and by using an aqueous solution which does not contain an organic solvent.

### SUMMARY OF THE INVENTION

An object of the present invention is to provide a method for preparing a lyophilized composition with improved stability and solubility, which comprises a first step of dissolving cyclophosphamide, sodium chloride, and D-mannitol in water as a solvent in a reaction container at 40° C. to 70° C.; and a second step of lyophilizing the solution obtained in the previous step.

Another object of the present invention is to provide a method for preparing a lyophilized composition with improved stability and solubility, which comprises a first step of dissolving 0.01 g to 0.1 g of cyclophosphamide per 1 mL of a solvent based on the weight of anhydrous cyclophosphamide, 40 parts by weight to 50 parts by weight of sodium chloride, and 30 parts by weight to 250 parts by weight of D-mannitol based on 100 parts by weight of cyclophosphamide in water for injection in a reaction container at 40° C. to 70° C.; a second step of sterilizing the solution obtained in the previous step with a membrane filter with a size of 0.2 μm or less followed by dispensing the solution into a container for injection; a third step of forming a lyophilized cake by lyophilizing the solution dispensed in the container; and a fourth step of vacuum-sealing the lyophilized composition in the container for injection.

Still another object of the present invention is to provide a lyophilized composition for injection, which comprises 6 parts by weight to 8 parts by weight of water, 40 parts by weight to 50 parts by weight of sodium chloride, and 30 parts by weight to 250 parts by weight of D-mannitol based on 100 parts by weight of cyclophosphamide.

To achieve the above objects, a first aspect of the present invention provides a lyophilized composition with improved stability and solubility, which comprises a first step of dissolving cyclophosphamide, sodium chloride, and D-mannitol in water as a solvent in a reaction container at 40° C. to 70° C.; and a second step of lyophilizing the solution obtained in the previous step.

A second aspect of the present invention is to provide a method for preparing a lyophilized composition with improved stability and solubility, which comprises a first step of dissolving 0.01 g to 0.1 g of cyclophosphamide per 1 mL of a solvent based on the weight of anhydrous cyclophosphamide, 40 parts by weight to 50 parts by weight of sodium chloride, and 30 parts by weight to 250 parts by weight of D-mannitol based on 100 parts by weight of cyclophosphamide in water for injection in a reaction container at 40° C. to 70° C.; a second step of sterilizing the solution obtained in the previous step with a membrane filter with a size of 0.2 μm or less followed by dispensing the solution into a container for injection; a third step of forming a lyophilized cake by lyophilizing the solution dispensed in the container; and a fourth step of vacuum-sealing the lyophilized composition in the container for injection.

A third aspect of the present invention is to provide a lyophilized composition for injection, which comprises 6 parts by weight to 8 parts by weight of water, 40 parts by weight to 50 parts by weight of sodium chloride, and 30 parts by weight to 250 parts by weight of D-mannitol based on 100 parts by weight of cyclophosphamide.

Hereinbelow, the present invention will be described in detail.

The present invention relates to a method for preparing a lyophilized cyclophosphamide composition, i.e., an insoluble drug, using water as a solvent for resolving the safety problem due to the residual solvent, instead of using an organic solvent such as butanol, etc., which has been conventionally used for the preparation of a lyophilized composition for injection of insoluble drugs. Further, the present invention is based on the discovery that cyclophosphamide, an insoluble raw material, is completely dissolved by maintaining a solution temperature to be 40° C. to 70° C., while also dissolving sodium chloride and D-mannitol, a conventional lyoprotectant, in an appropriate amount in order to completely dissolve cyclophosphamide in water. Furthermore, by lyophilizing the same, the lyophilized cyclophosphamide composition with improved stability and solubility compared to a lyophilized composition prepared by using a conventional organic solvent may be provided. Additionally, since the composition is prepared without using the organic solvent, it is safe against side effects caused by residual solvents, thereby confirming that the composition is appropriate to be used for injections.

In particular, when the lyophilized composition is provided in a vacuum-sealed form upon preparation thereof in an appropriate volume of a syringe container, it is advantageous in that such composition can be conveniently used in the field because it is rapidly dissolved when water for injection is added, compared to a lyophilized composition sealed by a conventional nitrogen-filling method.

The present invention provides a method for preparing a lyophilized composition with improved stability and solubility, which comprises a first step of dissolving cyclophosphamide, sodium chloride, and D-mannitol in water as a solvent in a reaction container at 40° C. to 70° C.; and a second step of lyophilizing the solution obtained in the previous step.

The amount of cyclophosphamide used for the preparation method of the present invention may be 0.01 g to 0.1 g per 1 mL of a solvent when the cyclophosphamide is taken as an anhydrous form. When the amount of the cyclophosphamide used is below 0.01 g/mL, an unnecessarily large amount of a solvent is used for producing a certain amount of a lyophilized cyclophosphamide composition. As a result, it causes the volume of a reactant to be increased, thereby decreasing the reaction efficiency. Alternately, when the amount of the cyclophosphamide exceeds 0.1 g/mL, it may lead to cyclophosphamide, a raw material, not being completely dissolved in a solution.

Specifically, the sodium chloride may be used in an amount of 40 parts by weight to 50 parts by weight based on 100 parts by weight of cyclophosphamide, but is not limited thereto. Addition of the sodium chloride may increase water solubility of insoluble cyclophosphamide. In particular, a lyophilized composition comprising sodium chloride should be rapidly dissolved when dissolved in an aqueous solution, e.g., water for injection, used for subsequent injections.

Lyophilization is a drying method involving freezing a sample in a solution state and removing moisture from the sample through sublimation by keeping the frozen sample under a reduced pressure. Accordingly, this method may be used for drying a sample containing water.

However, when the sample containing water is frozen, water molecules are crystallized during freezing while excluding media such as contaminants or solutes. Thus, ice crystals consisting only of water molecules are formed. Therefore, freeze concentration may occur since the solute in the aqueous material and the medium in the mixture are not diffused uniformly.

Accordingly, in order to overcome such problems, a method can be carried out by additionally adding lyoprotectants, which prevent physical and/or chemical damages and structural changes, to a solution. Generally used lyoprotectants include dimethylsulfoxide (DMSO), dextran, sucrose, glycerol, D-mannitol, sorbitol, fructose, trehalose, raffinose, serum albumin, etc., which may be used in combination according to a purpose. These lyoprotectants have been verified for bio-safety, but the mixing conditions satisfying the desired requirements are stringent. Therefore, the lyoprotectants have problems in that a preparation method is complicated and large manufacturing costs are required. The present inventors have confirmed that, among the lyoprotectants, D-mannitol is effective for preparing a lyophilized cyclophosphamide composition, and thereby used D-mannitol as a lyoprotectant.

Herein, the D-mannitol used as a lyoprotectant may be used in an amount of 30 parts by weight to 250 parts by weight based on 100 parts by weight of cyclophosphamide, but is not limited thereto.

Specifically, the cyclophosphamide may be used in a concentration of 0.01 g/mL to 0.1 g/mL, and the sodium chloride and the D-mannitol may be used in an amount of 40 parts by weight to 50 parts by weight and 30 parts by weight to 250 parts by weight based on 100 parts by weight of cyclophosphamide, respectively, but are not limited thereto.

Pure water for injection (WFI), saline, and buffer may be used as the water solvent. For example, since the lyophilized composition of the present invention comprises an adequate amount of sodium chloride based on the weight of cyclophosphamide contained therein, it can directly be injected into the body even when water for injection without salt is used to dissolve it at the concentration of cyclophosphamide to be administered.

Also, a sterilization step may additionally be comprised between the first and second steps. For example, the sterilization step may be carried out by filtering through a membrane below 0.2 μm. For example, since sterilization using the filtration of the membrane is achieved by excluding microorganisms depending on their size, it discharges contaminants larger than the pore size of the membrane surface. The membrane used for the present sterilization is a membrane with a 0.2 μm pore size, but is not limited thereto. In addition, nanofiltration membranes with 20 nm to 50 nm pore size may be used in order to further eliminate viruses.

On the other hand, the second step may be carried out by maintaining the solution cooled below −40° C. while maintaining the vacuum of 200 mTorr to 300 mTorr, but is not limited thereto. Additionally, it can be carried out by using a lyophilized method known in the art. Specifically, the second step can be carried out by maintaining the solution cooled below −40° C., maintaining the vacuum of 200 mTorr to 300 mTorr, and increasing a temperature in a phase while maintaining a certain time, but is not limited thereto. For example, a specific exemplary embodiment of the present invention cooled the solution at −40° C. while maintaining the same for 300 minutes. Thereafter, the resultant was decompressed to as low as 250 mTorr, and then a temperature was gradually increased to −15° C., 0° C., 10° C., and 25° C. At each temperature, this was maintained for 3120 minutes, 840 minutes, 600 minutes, and 240 minutes, respectively, thereby lyophilizing it.

Herein, the second step may be carried out in an injection container. As stated above, since cyclophosphamide, which is the lyophilized composition of the present invention, is relatively unstable in a solution state, it is preferable to immediately use cyclophosphamide upon preparation of the solution by adding injections. Therefore, in order to provide cyclophosphamide for injections, it is preferable to provide it in a syringe container by dispensing according to an amount per use. However, once a composition is lyophilized, it is difficult to exactly weigh and dispense the composition for an injection amount per use. Therefore, the composition is lyophilized by directly dispensing an adequate amount thereof to an injection container in a solution state so that the lyophilized composition containing a certain amount of cyclophosphamide can be easily prepared.

Specifically, a vacuum-sealing step may additionally be comprised after the second step. The vacuum-sealing step may be carried out using a conventional method known in the art. In addition, the pressure within a vacuum-sealed container may be maintained at 200 mTorr to 300 mTorr, but is not limited thereto. By maintaining the composition in a vacuum state after vacuum-sealing, as stated above, the composition may be dissolved by injecting a solution, e.g., water for injection, in order to be used for future injections. In such a case, it is advantageous in that it may rapidly dissolve the composition compared to conventional nitrogen-filled injections.

For example, the preparation method of the present invention may provide a lyophilized composition having a porosity of 40% to 90% prepared in the form of cake in a container for the injection.

Additionally, the lyophilized composition of the present invention with improved stability and solubility may be prepared through a process which comprises a first step of dissolving 0.01 g to 0.1 g of cyclophosphamide per 1 mL of a solvent based on the weight of cyclophosphamide, taken as an anhydrous form, 40 parts by weight to 50 parts by weight of sodium chloride, and 30 parts by weight to 250 parts by weight of D-mannitol based on 100 parts by weight of cyclophosphamide in water for injection in a reaction container at 40° C. to 70° C.; a second step of sterilizing the solution obtained in the previous step with a membrane filter with a size of 0.2 μm or less followed by dispensing the solution into a container for injection; a third step of forming a lyophilized cake by lyophilizing the solution dispensed in the container; and a fourth step of vacuum-sealing the lyophilized composition in the container for injection.

Further, the present invention may provide a lyophilized composition for injection, comprising an amount of 6 parts by weight to 8 parts by weight of water, 40 parts by weight to 50 parts by weight of sodium chloride, and 30 parts by weight to 250 parts by weight of D-mannitol based on 100 parts by weight of anhydrous cyclophosphamide.

The lyophilized composition of the present invention may be prepared according to the method in the first aspect of the present invention. Specifically, the lyophilized composition of the present invention may be prepared according to the method in the second aspect of the present invention, but is not limited thereto.

The lyophilized composition may be completely reconstituted within 60 seconds to 80 seconds when 50 mL of water for injection is added per 1000 mg of anhydrous cyclophosphamide.

The lyophilized composition of the present invention may be provided by vacuum-sealing in an amount corresponding to the fixed volume of an injection container, in which 10 mL to 50 mL of water for injection per 1000 mg of anhydrous cyclophosphamide may be added, so that the composition is easily used in the field, but is not limited thereto. For example, since the lyophilized composition of the present invention is provided to dispense an amount of a single dose in containers capable of holding an adequate volume of a solution used for a single dose according to the U.S. pharmacopeia, the composition can be injected by preparing the same as a solution with a desired final concentration by injecting a solution, such as water for injection, etc. For example, 200 mg, 500 mg, 1 g, and 2 g of a lyophilized composition may be provided by filling into a syringe container capable of holding an amount of 10 mL, 25 mL, 50 mL, and 100 mL of a solution, respectively, based on the weight of anhydrous cyclophosphamide, in order to provide the same with the final concentration of 20 mg/mL, but the composition is not limited thereto.

The lyophilized composition of the present invention, as stated above, may be provided in the form of a cake in a container for injection, wherein the composition has a porosity of 40% to 90%, but is not limited thereto.

The preparation method of the present invention can completely dissolve insoluble raw materials without using an organic solvent by adding a certain amount of D-mannitol and sodium chloride, which are lyoprotectants, followed by the dissolution of the D-mannitol and sodium chloride by increasing the temperature to a certain range, when preparing the solution of cyclophosphamide, an insoluble material. In addition, the preparation method of the present invention not only provides the composition with improved stability and solubility by lyophilizing the same, but also reduces risks that may occur due to residual organic solvents when using organic solvents. Further, the preparation method has an effect for reducing insoluble particles produced from the use of organic solvents. Furthermore, in a case in which it is prepared in a lyophilized formulation, the solubility is enhanced compared to conventional powdered products, and thus it shortens the time of dissolving active ingredients with the addition of water for injection. Accordingly, it provides convenience when actually used in the field.

### BRIEF DESCRIPTION OF THE DRAWINGS

FIG. 1 is a graph showing a result of insoluble particle counts measured for a lyophilized cyclophosphamide composition according to the present invention.

FIG. 2 is a graph showing a result of insoluble particle counts measured for a lyophilized cyclophosphamide composition (Comparison 3) prepared using t-butanol as a solvent instead of water for injection, as a comparative example of the present invention.

### DETAILED DESCRIPTION OF THE INVENTION

Hereinbelow, the present invention will be described in detail with accompanying exemplary embodiments. However, the exemplary embodiments disclosed herein are only for illustrative purposes and should not be construed as limiting the scope of the present invention.

### Example 1: Preparation of a Material Solution

A sample, in which cyclophosphamide monohydrate (1.069 g), sodium chloride (0.45 g), and D-mannitol (0.5 g) were mixed, was added to vials. Thereafter, water for injection (20 mL) was added to each vial having respective temperatures of 20° C., 30° C., 40° C., 50° C., 60° C., 70° C., and 80° C. In addition, the time was measured from the instant the water for injection was added to the instant the sample was completely dissolved. After being maintained for up to a total of 30 minutes, the mixture was filtered. The content of cyclophosphamide present in the filtrate was then measured. Additionally, the measured results are shown in Table 1 below.

TABLE 1

| Solubility | | | | | |
|---|---|---|---|---|---|
| temperature | | | | | Average |
| (° C.) | Sample | Test 1 | Test 2 | Test 3 | content |
| 20 | Sample 1 | Insoluble | Insoluble | Insoluble | 76.13 |
| 30 | Sample 2 | Partially soluble | Partially soluble | Partially soluble | 90.82 |
| 40 | Sample 3 | 8 min 50 sec | 8 min 36 sec | 8 min 58 sec | 100.29 |
| 50 | Sample 4 | 3 min 10 sec | 2 min 58 sec | 3 min 05 sec | 100.43 |
| 60 | Sample 5 | 1 min 25 sec | 1 min 42 sec | 1 min 10 sec | 100.29 |
| 70 | Sample 6 | 0 min 52 sec | 1 min 06 sec | 0 min 56 sec | 96.05 |
| 80 | Sample 7 | 0 min 37 sec | 0 min 30 sec | 0 min 32 sec | 76.48 |

The contents of Samples 1 to 7 were calculated by the following equations, respectively.

| Sample | Equation | Content |
|---|---|---|
| Sample 7 | 0.5313 × 25.3 mg/50 mL × 1.00/1.00 × 1.006 × 100 = | 76.48 |
| | 0.7071 × 1000.2 mg/20 mL × 2 mL/20 mL × 2 mL/20 mL | |
| Sample 6 | 0.6673 × 25.3 mg/50 mL × 1.00/1.00 × 1.006 × 100 = | 96.05 |
| | 0.7071 × 1000.3 mg/20 mL × 2 mL/20 mL × 2 mL/20 mL | |
| Sample 5 | 0.6966 × 25.3 mg/50 mL × 1.00/1.00 × 1.006 × 100 = | 100.29 |
| | 0.7071 × 1000.1 mg/20 mL × 2 mL/20 mL × 2 mL/20 mL | |
| Sample 4 | 0.6978 × 25.3 mg/50 mL × 1.00/1.00 × 1.006 × 100 = | 100.43 |
| | 0.7071 × 1000.4 mg/20 mL × 2 mL/20 mL × 2 mL/20 mL | |
| Sample 3 | 0.6969 × 25.3 mg/50 mL × 1.00/1.00 × 1.006 × 100 = | 100.29 |
| | 0.7071 × 1000.5 mg/20 mL × 2 mL/20 mL × 2 mL/20 mL | |
| Sample 2 | 0.6309 × 25.3 mg/50 mL × 1.00/1.00 × 1.006 × 100 = | 90.82 |
| | 0.7071 × 1000.2 mg/20 mL × 2 mL/20 mL × 2 mL/20 mL | |
| Sample 1 | 0.5289 × 25.3 mg/50 mL × 1.00/1.00 × 1.006 × 100 = | 76.13 |
| | 0.7071 × 1000.3 mg/20 mL × 2 mL/20 mL × 2 mL/20 mL | |

Additionally, in order to confirm a critical temperature showing the dramatic content change, a temperature in the range between 70° C. and 80° C., at which the content is dramatically reduced, was subdivided, and then the experiment was conducted in the same manner as above by using water for injection having a temperature of 72° C., 75° C., and 78° C., respectively. Further, the results are shown in Table 2 below.

TABLE 2

| Solubility | | | | | |
|---|---|---|---|---|---|
| temperature | | | | | Average |
| (° C.) | Sample | Test 1 | Test 2 | Test 3 | content |
| 78 | Sample 8 | 0 min 38 sec | 0 min 39 sec | 0 min 42 sec | 78.63 |
| 75 | Sample 9 | 0 min 45 sec | 0 min 53 sec | 0 min 45 sec | 89.39 |
| 72 | Sample 10 | 0 min 49 sec | 0 min 49 sec | 0 min 49 sec | 93.94 |

The contents of Samples 8 to 10 were calculated by the following equations, respectively.

| Sample | Equation | Content |
|---|---|---|
| Sample 8 | 0.5870 × 25.5 mg/50 mL × 261.09/279.10 × 1.006 × 100 = | 78.63 |
| | 0.6922 × 1000.0 mg/1000 mL × 25 mL/50 mL | |
| Sample 9 | 0.6446 × 25.5 mg/50 mL × 261.09/279.10 × 1.006 × 100 = | 89.39 |
| | 0.6922 × 1000.0 mg/1000 mL × 25 mL/50 mL | |
| Sample 10 | 0.6774 × 25.5 mg/50 mL × 261.09/279.10 × 1.006 × 100 = | 93.94 |
| | 0.6922 × 1000.0 mg/1000 mL × 25 mL/50 mL | |

### Example 2: Sterility Test after Sterile Filtration and Content Change Test

Similarly to Example 1, after sterilizing a solution prepared at various temperatures by a membrane filter having a pore size of 0.2 μm, the change in solution was confirmed by conducting the sterility test and by measuring the cyclophosphamide content. As a result, the solution was judged suitable from the sterility test upon the membrane filtration at all temperature conditions. Additionally, the change in the content of cyclophosphamide according to the sterilization process via a membrane filtration was hardly exhibited.

### Example 3: Mass Production of Lyophilized Composition for Injections

As shown in Table 3 below, raw materials of 1000 vial contents and vials were prepared.

TABLE 3

| | Amount of | | | | |
|---|---|---|---|---|---|
| | active | | | | |
| Raw material | material | Standard | Dose per vial | | Usage (g) |
| Cyclophos- | Anhydrous | USP | 1 | g | 1.069 |
| phamide | | | | | (monohydrate) |
| hydrate | | | | | |
| Sodium | | KP | 450 | mg | 450 |
| chloride | | | | | |
| D-mannitol | | KP | 500 | mg | 500 |
| Water for | | USP | 20 | mL | 20 L |
| injection | | | | | |
| (WFI) | | | | | |
| Vial (50 cc, | | KP | 1 | vial | 1000 |
| colorless) | | | | | |
| Vial cap | | — | 1 | unit | 1000 |
| Lyophilization | | KP | 1 | unit | 1000 |
| rubber | | | | | |
| stopper | | | | | |

As shown above, 1000 vials of cyclophosphamide hydrate, sodium chloride, and D-mannitol were prepared by dissolving the same in injection water. Specifically, the raw materials of the amount shown above were placed in a reaction container along with injection water (20 L), and they were dissolved while maintaining the reaction container at 40° C. to 70° C. Specifically, oxygen content was reduced by substituting with nitrogen for 10 minutes. Further, the raw materials of the amounts shown in Table 1 above and the injection water (about 18 L) with its temperature raised to be 60° C. were added in a preparation tank. After the mixture was completely dissolved by stirring, the injection water was further added so that the amount in the preparation tank reached 20 L. When it was confirmed that the raw materials were completely dissolved, the solution was no longer stirred.

The solution was filtered by applying a nitrogen pressure in the preparation tank containing the raw material solution (20 L) prepared as above. A filter integrity test was carried out by an integrity test (Sartocheck4 integrity tester) before and after the filtration, and the suitability was determined by measuring a value of bubble points. When the filtration was completed, it was transferred to a filling process.

Vial caps and filling components were washed using purified water and injection water, and these were sterilized in a high pressure steam sterilizer. The lyophilization rubber stopper was sterilized in a high pressure steam sterilizer, instead of washing it with a dust-free rubber stopper. The vials were sterilized using a tunnel sterilizer upon washing with an automatic vial washer. The solution (20 L) transferred to the filling process was dispensed to 1000 of the vials prepared above in equal amounts, respectively. Specifically, the solution was filled while the filling rate thereof was adjusted by arranging vials using a large-volume vial-filling machine. The solution was filled in an amount of 20 mL per vial, and rubber stoppers were half-capped for lyophilization. Once the filling was completed, the resultants were transferred to the lyophilization process.

The filled vials which were transferred to the lyophilization process were arranged in trays, and these were carefully placed into the chamber of a lyophilizer in sequential order of from the top rack to the bottom rack. After all trays were placed in the chamber, three temperature sensors within the chamber were placed inside of the vials in each rack. The tray alignment was confirmed again, and the gate of the lyophilizer was completely sealed. Thereafter, the vials were lyophilized according to the conditions shown in Table 4 below.

TABLE 4

| | | Decom- | | | | | | | | | Total time | |
|---|---|---|---|---|---|---|---|---|---|---|---|---|
| | Freeze | pression | 1 | 2 | 3 | 4 | 5 | 6 | 7 | 8 | Minute | hour |
| Rack | −40 | | −15 | −15 | 0 | 0 | 10 | 10 | 25 | 25 | | |
| temper- | | | | | | | | | | | | |
| ature | | | | | | | | | | | | |
| (° C.) | | | | | | | | | | | | |
| Time | 300 | | 120 | 3000 | 240 | 600 | 120 | 480 | 120 | 120 | 5100 | 85 |
| (minute) | | | | | | | | | | | | |
| Vacuum | — | 250 | 250 | 250 | 250 | 250 | 250 | 250 | 250 | 250 | | |
| (mTorr) | | | | | | | | | | | | |

Condenser set temperature: −40° C.

The lyophilization rubber stoppers were completely capped in a vacuum state using an automatic stopper device. An automatic sealing machine was adjusted to the height of the vials, and the vials were then sealed. Vials having a poorly sealed condition and rubber stoppers with a capped condition that is damaged were disposed. The lyophilized products were then transferred to a foreign inspection process. Specifically, the foreign inspection of the transferred products was carried out by the naked eye against black and white backgrounds under illumination having more than 1,000 lux directly under a white light source.

### Example 4: Usage of D-Mannitol and Change in Dissolution Time According to Vial-Filling Conditions

Vials for injections and lyophilized compositions were prepared with the contents shown in Table 5 below, respectively. In addition, the dissolution time was measured from the addition of injection water (50 mL) in each vial to the instant of complete dissolution thereof, and the results are shown in Table 6. The vials were adjusted to a vacuum state of 200 mTorr to 300 mTorr. The sample in Comparison 1 had the constitution corresponding to Sample 11, but the sample in Comparison 1 was Nitrogen-packed. Additionally, the sample of Comparison 2 had the conditions corresponding to Sample 11, but D-mannitol used in the sample of Comparison 2 was 0.1 g.

TABLE 5

| | Amount of raw material (g) | | | |
|---|---|---|---|---|
| | Cyclophosphamide | Sodium | D-man- | Injection water |
| | hydrate | chloride | nitol | (mL) |
| Sample 11 | 1.069 | 0.45 | 0.5 | 20 |
| Sample 12 | 1.069 | 0.45 | 2 | 20 |
| Compar- | 1.069 | 0.45 | 0.1 | 20 |
| ison 2 | | | | |

TABLE 6

| Sample | Dissolution time in accordance with filling conditions | | |
|---|---|---|---|
| (condition) | test 1 | test 2 | test 3 |
| Sample 11 | 1 min 4 sec | 1 min 15 sec | 1 min 09 sec |
| (Vacuum) | | | |
| Sample 12 | 1 min 8 sec | 1 min 1 sec | 1 min 16 sec |
| (Vacuum) | | | |
| Comparison 1 | 5 min 10 sec | 5 min 45 sec | 5 min 24 sec |
| (Nitrogen-packed) | | | |
| Comparison 2 | 6 min 15 sec | 6 min 32 sec | 6 min 20 sec |
| (Vacuum) | | | |

As shown in Table 6, although a lyophilized composition has the composition corresponding to Sample 11, it exhibited a significantly extended dissolution time when the composition was finally provided in a Nitrogen-packed state, instead of a vacuum state. Additionally, even if a lyophilized composition prepared with D-mannitol, of which the amount was reduced to 0.1 g, was stored in a vacuum state, it still required a remarkably extended dissolution time. It appears that the feature was caused by a phenomenon in which main ingredients are densely stuck due to the reduced amount of D-mannitol, a lyoprotectant, when forming a cake.

### Example 5: Measurement of Insoluble Particles

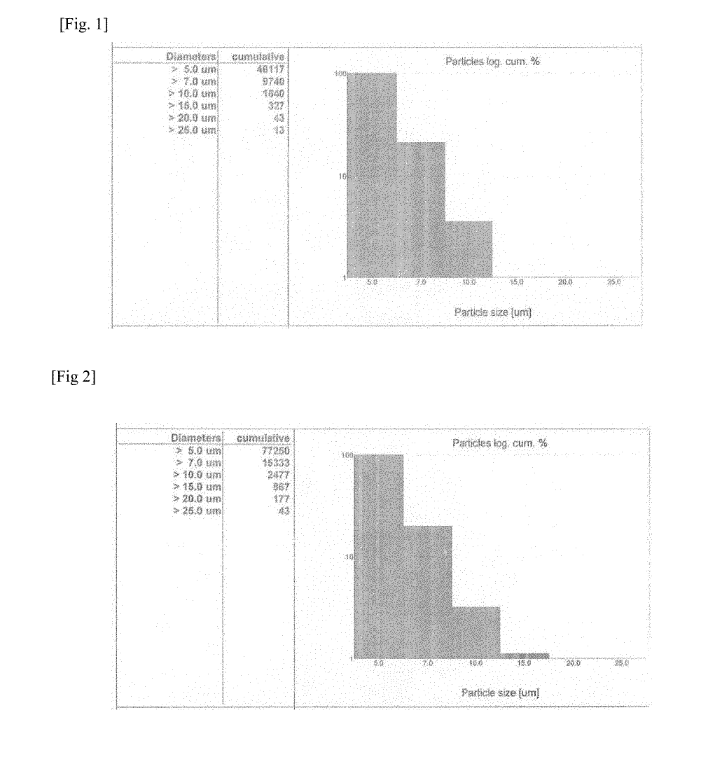

Insoluble particles existing among the prepared lyophilized composition were counted. The particles satisfied a standard tolerance, whereby less than 6000 particles per container should have a diameter greater than 10 μm, while less than 600 particles per container should have a diameter greater than 25 μm. The measurement results are shown in FIG. 1. Meanwhile, the measurement results of the lyophilized composition in Comparison 3 prepared by using t-butanol, instead of injection water, are shown in FIG. 2.

As shown in FIGS. 1 and 2, it was confirmed that the cumulative number of the insoluble particles having a size greater than 10 μm was 1640 per container in Sample 11 of the present invention, whereas Comparison 2, prepared using t-butanol, an organic solvent, contained 2477 insoluble particles, which was much greater than the cumulative number of particles in Sample 11.

### Example 6: Measurement of Porosity

A pore size of cyclophosphamide powders, a raw material, in Samples 11 and 12 (including 0.5 g and 2.0 g of D-mannitol), and Comparison 4 of the present invention was measured. Porosity measurement for the samples prepared by the present applicant was requested from the Korea Polymer Testing & Research Institute (KOPTRI), an authorized international testing agency. The measurement results for Samples 11 and 12, and Comparison 4 are shown in Table 7 below.

TABLE 7

| Sample | Analysis of item | Unit | Result |
|---|---|---|---|
| Sample 11 | Apparent density | g/cm³ | 0.59 |
| | Average diameter | μm | 0.43 |
| | Porosity | % | 57.8 |
| Sample 12 | Apparent density | g/cm³ | 0.32 |
| | Average diameter | μm | 14.8 |
| | Porosity | % | 80.9 |
| Comparison 4 | Apparent density | g/cm³ | 1.31 |
| | Average diameter | μm | 0.02 |
| | Porosity | % | 5.24 |

As shown in Table 7 above, it was confirmed that the cyclophosphamide powder, a raw material, exhibits a significantly low porosity because D-mannitol and sodium chloride, which refer to lyoprotectants, are not contained therein, thereby accordingly having a remarkably high density. These results show that the formulation is inappropriate for use in the field because it cannot easily be dissolved for injection water as raw materials therein are compactly condensed.

It will be obvious to those having skill in the art that many changes may be made to the details of the above-described embodiments without departing from the underlying principles of the invention. The scope of the present invention should, therefore, be determined only by the following claims.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A method for preparing a lyophilized composition with improved stability and solubility, which comprises:
a first step of dissolving cyclophosphamide, sodium chloride, and D-mannitol in water as a solvent in a reaction container at 40° C. to 70° C.; and
a second step of lyophilizing the solution obtained in the previous step.

2. The method of claim 1, wherein the cyclophosphamide, taken as an anhydrous form, is contained in an amount of 0.01 g to 0.1 g per 1 mL of water.

3. The method of claim 1, wherein sodium chloride is used in an amount of 40 parts by weight to 50 parts by weight based on 100 parts by weight of cyclophosphamide.

4. The method of claim 1, wherein D-mannitol is used in an amount of 30 parts by weight to 250 parts by weight based on 100 parts by weight of cyclophosphamide.

5. The method of claim 1, wherein cyclophosphamide is used in a concentration of 0.01 g/mL to 0.1 g/mL, and sodium chloride and D-mannitol are used in an amount of 40 parts by weight to 50 parts by weight and 30 parts by weight to 250 parts by weight based on 100 parts by weight of cyclophosphamide, respectively.

6. The method of claim 1, further comprising sterilizing the solution between the first step and the second step.

7. The method of claim 1, wherein the second step is carried out by maintaining the solution cooled below −40° C. while maintaining the vacuum of 200 mTorr to 300 mTorr.

8. The method of claim 1, further comprising vacuum-sealing after the second step.

9. The method of claim 1, wherein the lyophilized composition having a porosity of 40% to 90% is prepared in the form of a cake in a container for the injection.

10. A method for preparing a lyophilized composition with improved stability and solubility, which comprises:
a first step of dissolving 0.01 g to 0.1 g of cyclophosphamide per 1 mL of a solvent based on the weight of anhydrous cyclophosphamide, 40 parts by weight to 50 parts by weight of sodium chloride, and 30 parts by weight to 250 parts by weight of D-mannitol based on 100 parts by weight of cyclophosphamide in water for injection in a reaction container at 40° C. to 70° C.;
a second step of sterilizing the solution obtained in the previous step with a membrane filter with a size of 0.2 μm or less followed by dispensing the solution into a container for injection;
a third step of forming a lyophilized cake by lyophilizing the solution dispensed in the container; and
a fourth step of vacuum-sealing the lyophilized composition in the container for injection.

11. A lyophilized composition for injection, comprising an amount of 6 parts by weight to 8 parts by weight of water, 40 parts by weight to 50 parts by weight of sodium chloride, and 30 parts by weight to 250 parts by weight of D-mannitol based on 100 parts by weight of anhydrous cyclophosphamide.

12. The lyophilized composition of claim 11, wherein the lyophilized composition is prepared by the method of claim 1.

13. The lyophilized composition of claim 11, wherein the lyophilized composition is prepared by the method of claim 10.

14. The lyophilized composition of claim 11, wherein, upon addition of 50 mL of water for injection per 1000 mg of anhydrous cyclophosphamide, the composition is completely reconstituted within 60 seconds to 80 seconds.

15. The lyophilized composition of claim 11, wherein the lyophilized composition is provided vacuum-sealed in an amount corresponding to a container for injection with a predetermined volume enabling addition of 10 mL to 50 mL of water for injection per 1000 mg of anhydrous cyclophosphamide.
